(19) United States
(12) Patent Application Publication
(10) Pub. No.: US 2019/0275032 A1
(43) Pub. Date: Sep. 12, 2019

(54) USE OF ECM BIOMARKERS FOR DETERMINING THE TREATMENT ONSET WITH NINTEDANIB AND PIRFENIDONE

(71) Applicant: Boehringer Ingelheim International GmbH, Ingelheim am Rhein (DE)

(72) Inventors: Susanne STOWASSER, Loerzweiler (DE); Claudia DIEFENBACH, Stockach (DE)

(21) Appl. No.: 16/304,703

(22) PCT Filed: May 26, 2017
(86) PCT No.: PCT/EP2017/062734

§ 371 Date: Nov. 27, 2018

(30) Foreign Application Priority Data
May 27, 2016 (EP) 16 001 210.0
Sep. 2, 2016 (EP) 16 187 082.9

(51) Int. Cl. A61K 31/496; A61K 31/4418; A61P 11/00; G01N 33/68

(52) U.S. Cl. CPC A61K 31/496; G01N 33/6893; A61P 11/00; A61K 31/4418

(57) ABSTRACT

The invention relates to a method for treating idiopathic pulmonary fibrosis using a compound selected from the group consisting of nintedanib, or a pharmaceutically acceptable salt thereof, and pirfenidone, or a pharmaceutically acceptable salt thereof.

### FIELD OF THE INVENTION

The invention relates to a method for treating idiopathic pulmonary fibrosis using a compound selected from the group consisting of nintedanib and pharmaceutically acceptable salt thereof, and pirfenidone, and a pharmaceutically acceptable salt thereof

### BACKGROUND OF THE INVENTION

### IPF

Idiopathic pulmonary fibrosis (IPF) belongs to a large group of more than 200 lung diseases known as interstitial lung diseases (ILDs), which are characterized by the involvement of the lung interstitium, the tissue between the air sacs of the lung.

Idiopathic pulmonary fibrosis (IPF) is a rare disease of unknown aetiology that is characterized by progressive fibrosis of the interstitium of the lung, leading to decreasing lung volume and progressive pulmonary insufficiency. The course of the disease in individual patients is variable: some patients progress rapidly, others have periods of relative stability punctuated by acute exacerbations and others progress relatively slowly. Acute exacerbations of IPF are events of respiratory deterioration of unidentified cause that occur in 5-10% of patients annually and are associated with a very poor outcome. IPF is most prevalent in middle aged and elderly patients, and usually presents between the ages of 40 and 70 years. The median life expectancy in IPF patients after diagnosis is 2 to 3 years. The latest update on clinical practice guideline for the treatment of IPF, jointly issued in 2015 by the American Thoracic Society (ATS), European Respiratory Society (ERS), Japanese Respiratory Society (JRS) and Latin American Thoracic Association (ALAT) has provided a conditional recommendation for treatment with nintedanib or pirfenidone for the majority of IPF patients, taking into account individual patient values and preferences. Conventional IPF treatments such as n-acetylcysteine (NAC), corticosteroids, cyclophosphamide, cyclosporine and azathioprine are not approved treatments for IPF, and their efficacy is questionable or even harmful. Nonpharmacological therapies such as pulmonary rehabilitation and long-term oxygen therapy are recommended for some patients, but their efficacy in patients with IPF has not been established. Lung transplant has been shown to positively impact survival in patients with IPF. Although the number of patients transplanted due to IPF has increased steadily over the last years, the scarce availability of donor organs, as well as the comorbidities and advanced age preclude many patients from referral to lung transplant. Pirfenidone, a compound which demonstrated anti-fibrotic activity in non-clinical models, was first licensed in Japan in 2008 based on two local trials which showed a reduced decline of vital capacity under treatment with the compound. In the international Phase III CAPACITY program, pirfenidone demonstrated efficacy on the primary FVC lung function endpoint in only one of two confirmatory trials. The additional confirmatory ASCEND Phase III trial requested by FDA met the primary endpoint of change from baseline FVC % predicted. Pirfenidone is also licensed since February 2011 for the treatment of mild to moderate IPF in the European Union and since October 2014 for the treatment of IPF in the United States of America. It is also licensed in several other countries. Nintedanib is a small molecule intracellular tyrosine kinase inhibitor which has demonstrated anti-fibrotic and anti-inflammatory activity in preclinical models. The two replicate Phase III INPULSIS trials and the Phase II TOMORROW trial consistently showed positive results for the efficacy of nintedanib 150 mg twice daily versus placebo in patients with IPF. Both INPULSIS trials showed that nintedanib reduced the annual rate of decline in FVC (mL/year) by approximately 50%, consistent with slowing disease progression. Based on these three clinical trials, nintedanib was approved for the treatment of IPF in the USA in October 2014, in the European Union in January 2015 and in Japan in July 2015. As of 15 Apr. 2017, nintedanib has been authorised in the indication of treatment of IPF in sixty countries (including Canada, Switzerland, Russia, Australia, Chile, Ecuador and Taiwan),It has been submitted for marketing authorization in other countries across the world.

### Nintedanib

Nintedanib, the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone is an innovative compound having valuable pharmacological properties, especially for the treatment of oncological diseases, immunologic diseases or pathological conditions involving an immunologic component, or fibrotic diseases.

The chemical structure of this compound is depicted below as Formula A.

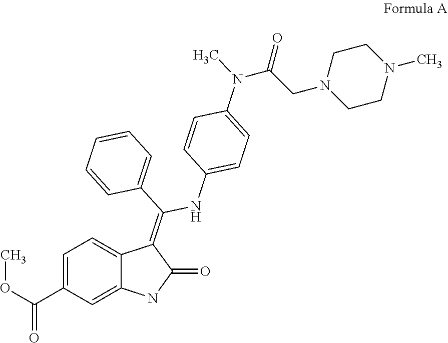 [SMILES: COC(=O)c1ccc2c(c1)NC(=O)/C2=C(\Nc1ccc(N(C)C(=O)CN2CCN(C)CC2)cc1)c1ccccc1]

The base form of this compound is described in WO 01/27081, the monoethanesulpho-nate salt form is described in WO 2004/013099 and various further salt forms are presented in WO 2007/141283. The use of this molecule for the treatment of immuno-logic diseases or pathological conditions involving an immunologic component is being described in WO 2004/017948, the use for the treatment of oncological diseases is being described in WO 2004/096224 and the use for the treatment of fibrotic diseases is being described in WO 2006/067165.

The monoethanesulphonate salt form of this compound presents properties which makes this salt form especially suitable for development as medicament.

The chemical structure of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate is depicted below as Formula A1.

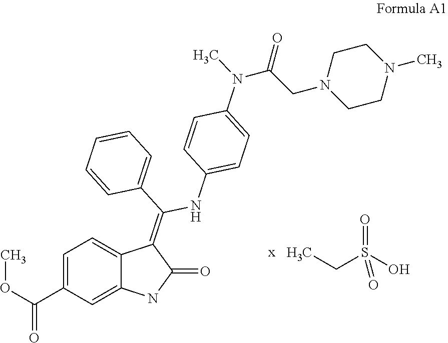

Preclinical studies have shown that this compound is a highly potent, orally bioavailable inhibitor of vascular endothelial growth factor receptors (VEGFRs), platelet-derived growth factor receptors (PDGFRs) and fibroblast growth factor receptors (FGFRs) and the antifibrotic potential of VEGFR, PDGFR, and FGFR inhibition with nintedanib has been evaluated in a series of preclinical studies. Nintedanib was shown to inhibit PDGFR-α and-β activation and proliferation of normal human lung fibroblasts in vitro and to inhibit PDGF-BB-, FGF-2-, and VEGF-induced proliferation of human lung fibroblasts from patients with IPF and control donors. Nintedanib attenuated PDGF- or FGF-2-stimulated migration of lung fibroblasts from patients with IPF and inhibited transforming growth factor (TGF)-β-induced fibroblast to myofibroblast transformation of primary human lung fibroblasts from IPF patients. In two different mouse models of IPF, nintedanib exerted anti-inflammatory effects as shown by significant reductions in lymphocyte and neutrophil counts in the bronchoalveolar lavage fluid, reductions in inflammatory cytokines and reduced inflammation and granuloma formation in histological analysis of lung tissue. IPF mouse models also revealed nintedanib-associated antifibrotic effects as shown by significant reductions in total lung collagen and by reduced fibrosis identified in histological analyses.

Posology: Nintedanib the recommended dose is 150 mg nintedanib twice daily administered approximately 12 hours apart. The amount of nintedanib to be administered is calculated on the free base while it is actually formulated as monoethanesulphonate. The 100 mg twice daily dose is only recommended to be used in patients who do not tolerate the 150 mg twice daily dose.

If a dose is missed, administration should resume at the next scheduled time at the recommended dose.

If a dose is missed the patient should not take an additional dose. The recommended maximum daily dose of 300 mg should not be exceeded. Dose adjustments: In addition to symptomatic treatment if applicable, the management of adverse reactions to nintedanib (see Ofev® EPAR of the EMA, sections 4.4 and 4.8) could include dose reduction and temporary interruption until the specific adverse reaction has resolved to levels that allow continuation of therapy. Nintedanib treatment may be resumed at the full dose (150 mg twice daily) or a reduced dose (100 mg twice daily). If a patient does not tolerate 100 mg twice daily, treatment with nintendanib should be discontinued. In case of interruptions due to aspartate aminotransferase (AST) or alanine aminotransferase (ALT) elevations >3× upper limit of normal (ULN), once transaminases have returned to baseline values, treatment with Ofev may be reintroduced at a reduced dose (100 mg twice daily) which subsequently may be increased to the full dose (150 mg twice daily) (see EPAR sections 4.4 and 4.8).

### Hepatic Impairment

Nintedanib is predominantly eliminated via biliary/faecal excretion (>90%). Exposure increased in patients with hepatic impairment (Child Pugh A, Child Pugh B; see EPAR section 5.2). In patients with mild hepatic impairment (Child Pugh A), the recommended dose of Ofev is 100 mg twice daily approximately 12 hours apart. In patients with mild hepatic impairment (Child Pugh A), treatment interruption or discontinuation for management of adverse reactions should be considered. The safety and efficacy of nintedanib have not been investigated in patients with hepatic impairment classified as Child Pugh B and C. Treatment of patients with moderate (Child Pugh B) and severe (Child Pugh C) hepatic impairment with Ofev is not recommended (see EPAR section 5.2).

### Pirfenidone

Pirfenidone is 5-methyl-1-phenyl-2(1H)-Pyridinone having the CAS number 53179-13-8. The chemical structure of this compound is depicted below as Formula B:

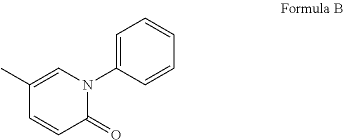

Pirfenidone is marketed as Esbriet® in capsules of 267 mg pirfenidone.

Esbriet is used to treat adults with mild to moderate idiopathic pulmonary fibrosis (IPF) in the EU.

### Treatment Regimen for Adults

Upon initiating treatment, the dose should be titrated to the recommended daily dose of nine capsules per day over a 14-day period as follows:
Days 1 to 7: one capsule, three times a day (801 mg/day)
Days 8 to 14: two capsules, three times a day (1602 mg/day)
Day 15 onward: three capsules, three times a day (2403 mg/day)

The recommended daily dose of Esbriet for patients with IPF is three 267 mg capsules three times a day with food for a total of 2403 mg/day. Doses above 2403 mg/day are not recommended for any patient.

Patients who miss 14 consecutive days or more of Esbriet treatment should re-initiate therapy by undergoing the initial 2-week titration regimen up to the recommended daily dose. For treatment interruption of less than 14 consecutive days, the dose can be resumed at the previous recommended daily dose without titration.

Although nintedanib and pirfenidone can be considered a standard of care for patients diagnosed with IPF, it remains unclear when to start and when to stop treatment with either of the drugs, given the unpredictability of clinical course in the individual patient, or in other words, which patients might benefit most from one of the antifibrotic treatments available. With the introduction of nintedanib in the treatment algorithm of IPF, there is an additional need to further characterize its profile in patients at an early disease stage, i.e. in patients with limited lung volume impairment, and to address the question when to start treatment in these patients. Currently, many physicians apply a wait and watch strategy for these patients as there are no markers to predict the individual course in a given patient or response to treatment which may result in a delay of treatment initiation. Identifying biomarkers to predict the clinical course and benefits of therapy for a given patient early in the course of the disease remains one of the most urgent and relevant challenges in patient management.

### SUMMARY OF THE INVENTION

One embodiment of the invention is a compound selected from the group consisting of nintedanib and pharmaceutical acceptable salts thereof, and pirfenidone and pharmaceutical acceptable salts thereof, for use in the treatment of idiopatic pulmonary fibrosis, wherein the onset of the treatment is determined by the determination of CRPM content of a body sample of the patient at least at two consecutive time points and wherein the treatment starts if the rate of the change of concentration of CRPM is greater than 1.7 ng/ml per month, more preferably 1 ng/ml per month, most preferred more than 0 ng/ml per month.

In a preferred embodiment of the invention the compound is nintedanib in the form of its monoethanesulphonate salt.

In a preferred embodiment of the invention the body sample is either plasma or serum.

The invention allows an early identification of those IPF patients that particularly benefit from the treatment because their disease will further progress.

### DETAILED DESCRIPTION OF THE INVENTION

### CRPM Determination

CRPM means C-reactive protein degraded by matrix metalloprotease ⅛ (MMP-⅛) that has been evaluated in the PROFILE study. In this study serum samples were prospectively collected at baseline, 1 month, 3 months, and 6 months and were analysed for a panel of novel matrix metalloprotease (MMP)-degraded ECM proteins, by ELISA-based, neoepitope assay. 11 neoepitopes were tested in a discovery cohort of 55 patients to identify biomarkers of sufficient rigour for more detailed analyses. Eight were then further assessed in a validation cohort of 134 patients with 50 age-matched and sex-matched controls. Changes in biomarker concentrations were related to subsequent risk of progression of idiopathic pulmonary fibrosis (defined as death or absolute decline in forced vital capacity >10% at 12 months after study enrolment) using a repeated measures model. The PROFILE study is registered on ClinicalTrials.gov, numbers NCT01134822 and NCT01110694, see JENKINS et al., Lancet Respir Med (2015), http://dx.doi.org/10.1016/S2213-2600(15)00048-X, page 1-11. This study revealed that for CRPM a rate of change (slope) greater than 0 ng/ml per month conferred a HR of 2.16 (95% Cl 1.15-4.07), whereas a rate greater than 1 ng/ml per month resulted in an HR 4.08 (2.14-7.8), and a rate greater than 1.7 ng/ml per month was associated with an HR 6.61 (95% Cl 2.74-15.94). Hazard ratio represents the mortality risk in participants with rising neoepitope concentrations relative to those with stable or falling concentrations (see, page 7, col. 2, 3rd paragraph of JENKINS et al.).

The C-reactive protein (CRP) is considered the prototypical acute phase reactant in

human and is produced in response to a variety of clinical conditions including infection, inflammation and tissue injury. During acute phase stimulus the serum concentration of CRP approaches a 1000 to 10.000-fold increase within 24-48 hours and decreases just as rapidly to the low normal concentration of a few μg/mL. CRP is upregulated in both situations of acute and chronic inflammatory diseases, however it is a non-specific biochemical marker due to its upregulation in all inflammatory diseases the prototypical acute phase reactant in human and is produced in response to a variety of clinical conditions including infection,

inflammation and tissue injury (VOLANAKIS, Mol Immunol 2001; 38: 189-97-DU CLOS, Ann Med 2000; 32: 274-8-HIRSCHFIELD, PEPYS, QJM 2003; 96:793-807).

The determination of CRPM in serum samples follows the procedure disclosed in SKJ∅T-ARKIL et al., Clinical and Experimental Rheumatology 2012; 30: 371-379, in particular 373-375:
“In vitro cleavage of CRP
Purified CRP from human serum (Alpha Diagnostics) was cleaved with MMP-1, MMP-9, cathepsin K, cathepsin S (Calbiochem, VWR), MMP-3, MMP-8 (Abcam), A Disintegrin And Metalloproteinase with a Thrombospondin motif (ADAMTS)-1, and -8 (Abnova). The proteases were
activated according to the manufacturers's instructions. Each cleavage was
performed separately by mixing 200 μg CRP and 2 μg of activated enzymes in
MMP buffer (100 mM Tris-HCl, 100 mM NaCl, 10 mM CaCl₂, 2 mM ZnOAc, pH
8.0), cathepsin buffer (50 mM NaOAc, 20 mM L-cystine, pH=5.5) or aggrecanase
buffer (50 mM tris-HCl, 10 mM NaCl, 10 mM CaCl2, pH=7.5). As control 200 μg CRP was mixed with MMP buffer only. Each aliquot was incubated for three days at 37° C. All MMP cleavages were terminated using GM6001 (Sigma-Aldrich) and all cathepsin and aggrecanase cleavages using E64 (Sigma-Aldrich). Finally the cleavage was verified by visualization using the SilverXpress® Silver Staining Kit (Invitrogen) according to the manufacturers' instructions.

### Peptide Identification by MS

The cleavage products were purified and desalted using reversed phase (RP)

micro-columns (Applied Biosystems) prior to nanoLC-MS-MS analysis as describes in literature (see THINGHOLM & LARSEN: Methods Mol Biol 2009; 527: 57-66, xi.28). The purified peptides were resuspended in 100% formic acid, diluted with H₂O and loaded directly onto a 18 cm RP capillary column using a nano-Easy-LC system (Proxeon, Thermo Scientific). The peptides were eluted using a gradient from 100% phase A (0.1% formic acid) to 35% phase B (0.1%

formic acid, 95% acetonitrile) over 43 min directly into an LTQ-Orbitrap XL mass spectrometer (Thermo Scientific).

For each MS scan (Orbitrap, resolution of 60000, 300-1800Da range) the five

most abundant precursor ions were selected for fragmentation (CID). The raw data files were converted to mgf files and searched in Mascot 2.2 using

Proteome Discoverer (Thermo Scientific).

Peptides with a mascot probability score p<0.05 were further analysed.

### Selection of Peptide for Immunisations

The first six amino acids of each free end of the sequences identified by MS

were regarded as a neoepitope generated by the protease in question. All protease-generated sequences were analysed for homology and distance to

other cleavage sites and then blasted for homology using the NPS@: network

protein sequence analysis (COMBET, BLANCHET, GEOURJON, DELEAGE, Trends Biochem Sci 2000;25: 147-50).

### Immunisation Procedure

Six 4-6 week old Balb/C mice were immunised subcutaneously in the abdomen

with 200 μL emulsified antigen (50 μg per immunisation) using Freund's incomplete adjuvant (KAFVFPKESD-GGC-KLH and GNFEGSQSLV-GGC-OVA (Chinese Peptide Company, Beijing, China)). Immunisations were continued until stable titer levels were obtained. The mouse with the highest titer was selected for fusion and boosted intravenously with 50 μg immunogen in 100 μL 0.9% sodium chloride solution three days before isolation of the spleen for cell fusion. The fusion procedure has been previously described (GEFTER, MARGULIES, SCHARFF, Somatic Cell Genet 1977; 3: 231-6).

### Characterization of Clones

The potential sequences KAFVFP and GNFEGS, named CRP-MMP and CRPCAT respectively, were selected for antibody generation. Native reactivity

and peptide binding of the monoclonal antibodies were evaluated by displacement of human serum in a preliminary indirect ELISA using biotinylated peptides (KAFVFPKESD-K-Biotin or GNFEGSQSLV-K-Biotin) on a streptavidin coated microtitre plate and the supernatant from the growing monoclonal hybridoma. Tested were the specificities of clones to the free peptide (KAFVFPKESD or GNFEGSQSLV), a non-sense peptide, and the elongated peptide (RKAFVFPKESD or GGNFEGSQSLV).

Isotyping of the monoclonal antibodies was performed using the Clonotyping System-HRP kit (Southern Biotech). The selected clones were purified using Protein G columns according to manufacturer's instructions (GE Healthcare Life Science).

### Assay Protocol

The selected monoclonal antibodies were labelled with horseradish peroxidase

(HRP) using the Lightning link HRP labelling kit according to the instructions of the manufacturer (Innovabioscience). A 96-well streptavidin plate was coated with 1.25 ng/mL KAFVFPKESD-K-Biotin (CRP-MMP assay) or 0.40 ng/mL GNFEGSQSLVK-Biotin (CRP-CAT assay) dissolved in assay buffer (25 mM Tris, 1% BSA, 0.1% Tween-20, pH 7.4) and incubated 30 minutes at 20° C. 20 μL of free peptide calibrator or sample were added in duplicates to appropriate wells, followed by 100 μL of conjugated monoclonal antibody (1A7-HRP or 3H8-HRP) and incubated 1 hour at 20° C. Finally, 100 μL tetramethylbenzinidine (TMB) (Kem-En-Tec) was added and the plate was incubated 15 minutes at 20° C. in the dark. All the above incubation steps included shaking at 300 rpm. After each incubation step the plate was washed five times in washing buffer (20 mM Tris, 50 mM NaCl, pH 7.2). The TMB reaction was stopped by adding 100 μL of stopping solution (1% HCl) and measured at 450 nm with 650 nm as the reference. A master calibrator, prepared from the synthetic free peptide accurately quantified by amino acid analysis, was used as a calibration curve and plotted using a 4-parametric mathematical fit model.

### Technical Evaluation and Specificity

From 2-fold dilutions of quality control (QC) serum samples, linearity was calculated as a percentage of recovery of the 100% sample. The lower limit of detection was determined from 21 zero samples (i.e. buffer) and calculated as the mean +3× standard deviation. The inter- and intra-assay variation was determined by 12 independent runs of 8 QC samples, with each run consisting of two replicas of double determinations.

The stability of serum samples was measured for three samples, which have been frozen and thawed for one to ten times. The developed CRP-MMP and CRPCAT ELISAs were evaluated using the materials described under “In vitro cleavage”, where CRP was cleaved by different MMPs, cathepsins and aggrecanases. The materials were diluted 1:10 in the ELISA.

### CRP-MMP, CRP-CAT vs. Total CRP in Patients

CRP-MMP, CRP-CAT and full-length human CRP (Quantikine, R&D System) were assessed in serum from patients diagnosed with AS and compared to healthy sex- and age-matched controls from the Department of Medicine 3 of the University of Erlangen-Nuremberg.

Serum samples were retrieved from patients diagnosed with ankylosing spondylitis (AS) according to the modified New York criteria and from sex- and age-matched non-diseased controls. BASDAI and mSASSS was registered for the each of the AS patients.

The samples were diluted 1:4 in the CRP-MMP assay and in the CRP-CAT assay. The study was approved by the Ethics Committee of the University of Erlangen-Nuremberg and conformed to the principles outlined in the Declaration of Helsinki. Written informed consent was obtained from each person.

### Statistics

Serum levels of the individual biomarkers between AS patients and non-diseased controls were compared using two-sided non-parametric Wilcoxon rank sum test. Correlations between the biomarkers were investigated by non-parametric Spearman's test. Area under the curve was measured with use of Receiver Operating Characteristic (ROC). The biomarkers were investigated in odds ratios (extrapolated from weighted levels: lowest value in the population was set at 0 and highest at 1) where all subject were classified as having normal (within SD of the mean of the normal population) or high (>SD) levels of the biomarker. Results were considered statistically significant if p<0.05.”

The upper and lower limits of the quantification of CRPM (MMP degraded CRP-⅛) are 3.2 and 110.0 ng/ml, respectively and the intra/inter assay variability is 11.1% and 20.8% (JENKINs et al., Supplementary Table and Figure Legends).

### EXAMPLES

### A) Effect of Nintedanib on Biomarkers of ECM Turnover in Patients With IPF and Limited FVC Impairment

A 12-week, double blind, randomised, placebo controlled, parallel group trial followed by a single active arm phase of 40 weeks evaluating the effect of oral nintedanib 150 mg twice daily on change in biomarkers of extracellular matrix (ECM) turnover in patients with idiopathic pulmonary fibrosis (IPF) and limited forced vital capacity (FVC) impairment and to investigate the predictive value of change in those ECM biomarkers on disease progression.

Main Inclusion criteria: Male or female patients aged ≥40 years at Visit 1 (screening); IPF diagnosis based upon ATS/ERS/JRS/ALAT 2011 guideline within 3 years of Visit 0; HRCT performed within 18 months of Visit 0; confirmation of diagnosis by central review of chest HRCT and surgical lung biopsy (later if available) prior to randomisation; FVC 80% predicted of normal at Visit 1 (screening).

Posology: 300 mg daily (150 mg bid) with possibility to reduce total daily dose to 200 mg (100 mg bid) to manage adverse events (AEs).

Primary Endpoint: Rate of change (slope) in blood CRPM from baseline to week 12.

Key Secondary Endpoint: Proportion of patients with disease progression as defined by absolute FVC (% predicted) decline 0% or death until week 52.

Secondary Endpoints: Rate of change (slope) in blood Cl M from baseline to week 12;

Rate of change (slope) in blood C3M from baseline to week 12.

Further Endpoints (selected): Rate of change (slope) in blood CRPM, C1M and C3M from week 12 to week 52.

Safety criteria: Adverse events (especially SAE and other significant AE), physical examination, weight measurements, 12 lead electrocardiogram, vital signs and laboratory evaluations.

Statistical methods: Random coefficient regression models for continuous endpoints, Log rank tests, Kaplan-Meier plots and Cox regressions for time to event endpoints, logistic regression models or other appropriate methods for binary endpoints.

1. A method of treating idiopathic pulmonary fibrosis, the method comprising administering to a patient in need thereof a compound selected from the group consisting of nintedanib and a pharmaceutical acceptable salt thereof, and pirfenidone, and a pharmaceutical acceptable salt thereof, wherein the onset of the treatment is determined by the determination of C-reactive protein degraded by matrix metalloprotease ⅛ (CRPM1 content of a body sample, of the patient at least at two consecutive time points and wherein the treatment starts if the rate of the change of concentration of CRPM is greater than 0 ng/ml per month.

2. The method of claim 1, wherein the rate of the change of concentration of CRPM is greater than 1 ng/ml per month.

3. The method of claim 1, wherein the rate of the change of concentration of CRPM is greater than 1.7 ng/ml per month.

4. The method of claim 1, wherein nintedanib is in the form of its monoethanesulphonate salt.

5. The method of claim 1, wherein the body sample is serum.

6. The method of claim 1, wherein the body sample is plasma.

7. The method of claim 1, wherein the rate of the change of concentration of CRPM is determined on the basis of a time interval of 4 to 12 weeks.

8. The method of claim 1, wherein the rate of the change of concentration of CRPM is determined on the basis of a time interval of about 12 weeks.

9. (canceled)

10. The method of claim 1, wherein the body sample is blood.
